(19) United States
(12) Patent Application Publication
(10) Pub. No.: US 2016/0264344 A1
(43) Pub. Date: Sep. 15, 2016

(54) Stable Pressurized System Including Plastic Container And Active(s)-Containing Composition

(71) Applicant: S. C. Johnson & Son, Inc., Racine, WI (US)

(72) Inventors: Paul A. Clark, Racine, WI (US); Kimberly J. Harris, Milwaukee, WI (US); Peter N. Nguyen, Racine, WI (US); Rachel E. Salowitz, Whitefish Bay, WI (US); Bhaveshkumar Shah, Kenosha, WI (US); Maciej K. Tasz, Racine, WI (US); John S. Trent, Franklin, WI (US)

(73) Assignee: S. C. Johnson & Son, Inc., Racine, WI (US)

(21) Appl. No.: 15/094,542

(22) Filed: Apr. 8, 2016

Related U.S. Application Data
(63) Continuation-in-part of application No. 13/543,194, filed on Jul. 6, 2012.
(60) Provisional application No. 61/457,925, filed on Jul. 8, 2011.

(51) Int. Cl. B65D 83/38; B01F 3/04; A61L 9/14; A01M 1/20; C11B 9/00; B65D 83/28

(52) U.S. Cl. CPC B65D 83/38; C11B 9/00; B65D 83/28; A61L 9/14; A01M 1/2038; B01F 3/04056; B01F 2215/009

(57) ABSTRACT

Pressurized compositions propelled by a pressurizing component (preferably a propellant such as compressed gas) and further including at least one active ingredient, at least one high molecular weight nonionic surfactant, and water which is a major component of the composition and is at least substantially ion-free and/or salt-free (preferably ion-free and salt-free), wherein the composition is stored in and dispensed from a plastic pressurized container. The plastic is preferably PET or PEN. The composition has a pH of about 4 to about 8.5. The composition can include as active ingredient(s), one or more of a fragrance component, an odor eliminating compound, an insecticide, an antimicrobial, and a disinfectant.

### RELATED APPLICATIONS

This application is a continuation-in-part of and claims the benefit of U.S. Non-Provisional application Ser. No. 13/543,194 filed Jul. 6, 2012, which claims benefit of U.S. Provisional Application No. 61/457,925, filed Jul. 8, 2011, each above identified application being incorporated herein by reference.

### FIELD OF INVENTION

The invention is directed to a pressurized storage and dispensing system including a plastic container and a pressurizing component, and an active(s)-containing composition, wherein the composition is stored in and dispensed from the plastic container. The composition includes at least one pressurizing component (preferably compressed gas), at least one high molecular weight nonionic surfactant, at least one active ingredient, and water. The active(s)-containing composition is stable and preferably transparent (clear and colorless or colored) or translucent, and provides compatibility with and stability of the plastic container, by being at least substantially ion-free or salt-free to thereby avoid the occurrence and growth of crazes and/or stress cracks in a surface of the plastic container. The compositions may also have improved or enhanced intensity and longevity of the active component(s) therein.

### BACKGROUND OF THE INVENTION

Pressurized products, which include a compressed gas propellant, for dispensing fragrances and odor treating agents are known in the art. Conventionally, pressurized compositions are stored and dispensed from metal containers, such as aluminum or steel containers. In steel containers, in particular, corrosion is a concern primarily due to the high water content of the compositions stored in the container. As a result, the interior of the steel containers is usually coated and/or lined with polymeric coatings and conventionally the composition contains corrosion inhibiting compound(s). In containers storing compressed gas compositions, corrosion control and lining delamination have been a problem. In the absence of controlling corrosion and delamination, fragrance component(s) present in the composition may migrate into the coating of the container. An alternative to metal containers would, therefore, be beneficial.

Plastic containers would be a desirable alternative to metal containers for both manufacturers and consumers for packaging gas pressurized compositions. Plastic is desirable for manufacturers due to the lower weight of the product for transportation and stocking purposes, as well as the versatility of design configuration of a plastic container which allows for greater and varying aesthetic appeal of the product. Consumers also like the lightweight feel of plastic containers and varying aesthetics of plastic containers. Plastic containers can be transparent or translucent allowing the consumer to see the composition stored in the container. A clear product is perceived as desirable as an indication of purity. In prior art devices, however, plastic containers, when used as pressurized dispensing containers, had negative effects as to the stability of both the composition contained in the plastic container and the plastic container itself. The composition can discolor due to instability of the composition or container. The container can fail due to crazing or formation of stress cracks in wall surfaces of the container. Fragrances are one of the major factors in increasing cracking in a plastic container. Accordingly, a stable plastic pressurized dispensing container for fragrance-containing compositions under high pressure, for example as provided by compressed gas in the composition, would be advantageous and desirable.

Further to the above, in particular when the active includes a fragrance component, and whether for applying to the air or a fabric or other surface to achieve fragrancing, or treating air or fabric or other surface to eliminate an odor therein or thereon, it is desirable to provide the active component of the composition at a certain level or intensity over a length of time that allows the benefits to be appreciated for an extended period of time. Previously, it had been believed that larger aerosol particle sizes of a fragrance or odor treating composition were not desirable or as advantageous as small particle sizes because the particles of the composition would fall out of the air faster and result in wetting of the surfaces upon which the particles fall. Since higher spray rates generally provide larger particle sizes, higher spray rates were also considered not to be desirable. It would also be advantageous to provide the intended intensity and minimum fall out with a low level of or without the inclusion of an alcohol (which in the past has been included to aid in evaporation and fragrance lift) in a composition since alcohol undesirably results in a higher volatile organic compound (VOC) content in the composition.

### SUMMARY OF THE INVENTION

An active(s)-containing pressurized composition, in particular a pressurized composition containing at least one active ingredient (preferably a fragrance component), is provided in combination with a plastic aerosol container. The plastic aerosol container serves to store and dispense the composition. The plastic aerosol container and active(s)-containing composition in combination have a synergistic effect and compatibility which provides a pressurized, non-flammable, low volatile organic compound (VOC) content composition in a plastic container. The plastic of the body of the container can be clear and the composition can be provided as transparent or translucent at room temperature (21° C./70° F.) to provide a stable and aesthetically pleasing product. To avoid crazing or stress cracking of surfaces (e.g. wall surfaces) in the plastic container, which has been a primary flaw to overcome when providing a pressurized composition in a plastic container (especially when the composition includes a fragrance component), the composition is at least substantially ion-free or substantially salt-free (hereafter “substantially ion-free/salt-free), i.e., no added ions or salts, includes a high molecular weight nonionic surfactant, and additionally has a pH of about 4 to about 8.5. Ion(s) or ionic compounds, such as salt(s), refer to a cation, e.g., Na⁺, itself or as an element of a compound. If the ion is present as an element of a compound, the weight percent referred to is of the compound containing the ion.

More particularly, a preferred pressurized active(s)-containing composition is phase stable, single phase and will have the following features, which are further described below: (1) is substantially ion-free/salt-free or is ion-free/salt-free, (2) has a pH of about 4 to about 8.5, (3) has a low to zero VOC content, (4) is non-flammable, i.e., less than a total of 1% flammable ingredients and has a flash point above 60° C. (140° F.). Under Consumer Product Safety Commission (CPSC) regulations (see 16 CFR 1500.3(c) (6) (vii)-(viii), the criteria for non-flammable self-pressurized are as follows:
(vii) Extremely flammable contents of self-pressurized container means contents of a self-pressurized container that, when tested by the method described in §1500.45, a flashback (a flame extending back to the dispenser) is obtained at any degree of valve opening and the flashpoint, when tested by the method described in §1500.43a is less than 20° F. (−6.7° C.)
(viii) Flammable contents of self-pressurized container means contents of a self-pressurized container that, when tested by the method described in §1500.45, a flame projection exceeding 18 inches is obtained at full valve opening, or flashback (a flame extending back to the dispenser) is obtained at any degree of valve opening.
   Ultimately for a self-pressurized product to be considered non-flammable the material would need to not flashback to the valve, have a flashpoint >20° F., and a flame extension of <18° F. The only hazard associated with the product is that the contents are under pressure.

Alternatively, under Globally Harmonized System of Classification and Labeling of Chemicals (GHS) classification, a “non-flammable aerosol” would be considered Category 3 if it contains 1% flammable component and has a heat of combustion of ≦20 kiloJoule per gram (kJ/g); alternatively, if the aerosol passed all testing requirements (ignition distance test, heat of combustion, and enclosed space ignition test), then it would be considered Category 3, such Category 3 not requiring a GHS pictogram, “warning” as a signal word, or the hazard statement of “pressurized container: may burst if heated”, which is the equivalent to a non-flammable aerosol rating.

More preferably, the compositions may also include the following additional features: (5) is transparent or translucent at room temperature (21° C./70° F.), (6) includes only “generally regarded as safe” (GRAS) components, (7) is under an initial pressure of about 80 to about 150 psig (pounds per square inch gauge) at 50° C. (122° F.) and (8) has acceptable spray characteristics. To provide a single phase composition, a surfactant alone or in combination with a suitable non-water co-solvent (i.e., a co-solvent other than water) will be included in the composition. The surfactant is at least one high molecular weight nonionic surfactant, i.e., a nonionic surfactant having more than 8 carbon atoms therein. The high molecular weight nonionic surfactant serves an important role in providing stability between the composition and the plastic container holding the composition. Without being bound by theory, it is believed that all surfactants tend to go toward surfaces which than increases the possibility of interaction between the surfactant and the surface, e.g., a wall surface of a container, and cracking. So the present composition of the invention provides a system which is more homogeneous so as to prevent the surfactant from going into contact with surfaces. Lower molecular weight surfactants tend to attack plastic. In addition to moving the surfactant away from interaction with the plastic surfaces, the high molecular weight nonionic surfactant also provides significant action in the solubilization of the fragrance component thereby further adding to the stabilization of the combined composition and plastic container.

The pressurized composition includes as essential components at least one pressurizing component (preferably a propellant which in turn is preferably a compressed gas), at least one high molecular weight nonionic surfactant, at least one active ingredient (preferably at least one fragrance component and, optionally, one or more additional active ingredient, e.g., odor elimination component(s), insecticide(s), disinfectant(s), antimicrobial(s), or mixtures thereof), and substantially ion-free/salt-free or ion-free/salt-free water as a solvent carrier. Due to storage of the composition in a plastic container, the water is to be deionized water or other purified water that is substantially ion-free/salt-free or ion-free/salt-free since it was found that the presence of ions and/or salts affect the stability of the plastic container as described further below. The composition may optionally also include one or more adjuvants known for inclusion in such compositions. However, since the composition of the invention is used with a plastic dispensing container, ion- or salt-containing adjuvant compounds are not desirable for inclusion, i.e., preferably should be excluded from the composition.

A further manner of determining and controlling compatibility of the composition with the plastic container is through the use of Hansen Solubility Parameters (HSP) to determine compatible material(s) (e.g., both in terms of co-solvents and the aqueous solvent carrier) and polymer resin (the solvent(s) being in the composition and the polymer resin providing the storage portion, e.g., the body, of the container). HSP are used to determine Rₐ values and control solvent-polymer interactions so as to minimize or avoid crazing or stress-cracking of the polymer. HSP can be used to predict solvents which will dissolve or not into the plastic. A large difference in HSP (Rₐ), which is distance in Hansen space, will predict that a given solvent (e.g., water as a solvent carrier) will not likely dissolve into the plastic. If a co-solvent is present to further increase solubilization of active component(s), such will have a Hansen Solubility Parameters value as between the co-solvent and the polymer resin providing the plastic body of the container which has a difference which is indicative of the solvent(s) not dissolving into the polymer resin and thus not likely being subject to crazes or stress cracks. HSP are a way of predicting whether one material, such as a solvent, will dissolve into or have partial solubility with respect to another material, such as a polymer, and form a solution. Liquids with similar solubility parameters will be miscible, and polymers will dissolve in solvents whose solubility parameters are not too different from their own. While hydroxyl-containing compounds may be present as a co-solvent, e.g., in the form of an alkylene glycol or the like, an alkyl alcohol, while functional, is not generally desirable for inclusion since these materials increase the VOC content of the composition and since non-flammability is a desired characteristic of the composition being stored and dispensed from the plastic container. If an alkyl alcohol is present, such can be present only in a minimal amount which allows the composition to be non-flammable (i.e., as described above) and having an acceptable VOC content. HSP is further described below.

Further, the composition can be provided with improved intensity of the active(s) over an extended period of time by controlling certain other features of the composition and dispensing. The composition is specially formulated for dispensing from a suitable plastic container by a propellant, preferably a compressed gas propellant. The improvement experienced by a consumer of enhanced intensity of one or more actives in an environment of use is in terms of at least freshness, longer lasting, and consistency of quality or character between initial and terminal use, which each provide for a greater product impact on the user of the composition or product.

The invention allows for the use of any conventional or known active, such as to provide an air or fabric or other surface fragrance or odor treatment, insecticidal, disinfecting or antimicrobial composition, and yet provides enhancement of the active's properties, e.g., fragrance and/or odor elimination, with these conventional actives. This is achieved by maintaining or controlling certain properties of the composition present during dispensing of the product, i.e., spray rate and particle size.

BRIEF DESCRIPTION OF DRAWING

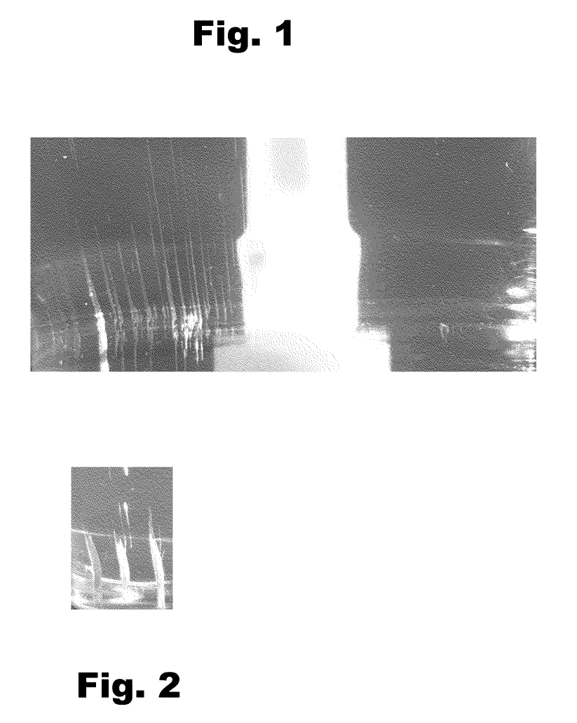

FIG. 1 shows stress micro-cracks present in a polyethylene terephthalate (PET) plastic.

FIG. 2 shows stress cracks in a PET plastic which are deeper than the micro-cracks of FIG. 1.

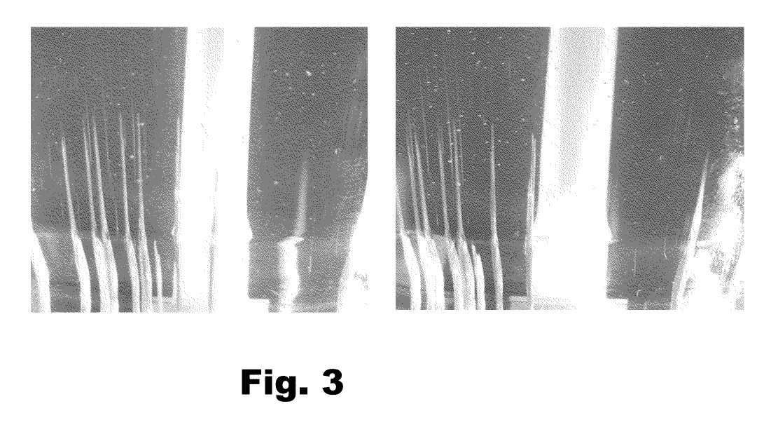

FIG. 3 shows cracks extending through the wall thickness of a plastic container which are large enough for the composition stored in the container to wick through.

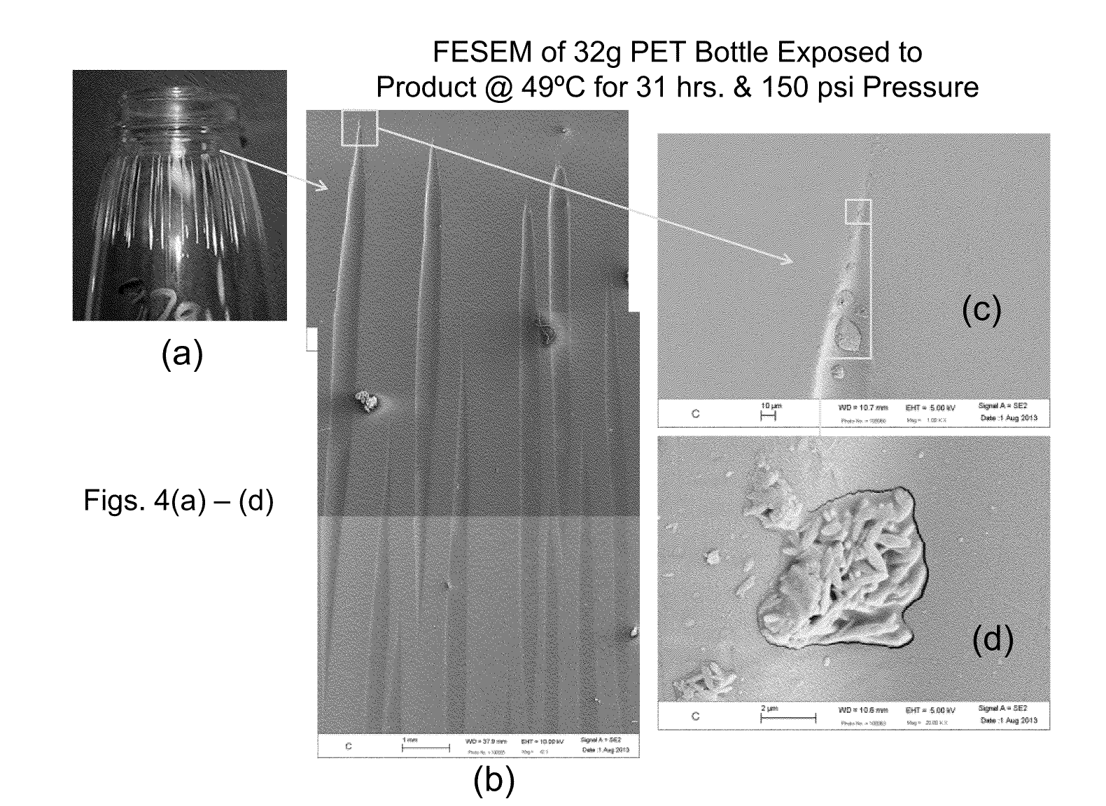

FIGS. 4(a)-(d) show different magnifications obtained using Field Emission Scanning Electron Microscopy (FESEM) of stress cracks in a 32 gram PET bottle containing a fragrance-containing composition at 49° C. (120° F.) for 31 hours under pressure at 150 psig. FIG. 4(a) shows stress cracks located in a wall in the neck area of the container. FIG. 4(b) is a more magnified view of a portion of the stress cracks of FIG. 4(a). FIG. 4(c) is a yet further magnified view of a tip of one stress crack of FIG. 4(b). FIG. 4(d) is a yet further magnified view of a portion of the tip of the stress crack of FIG. 4(c) showing salt compounds present therein.

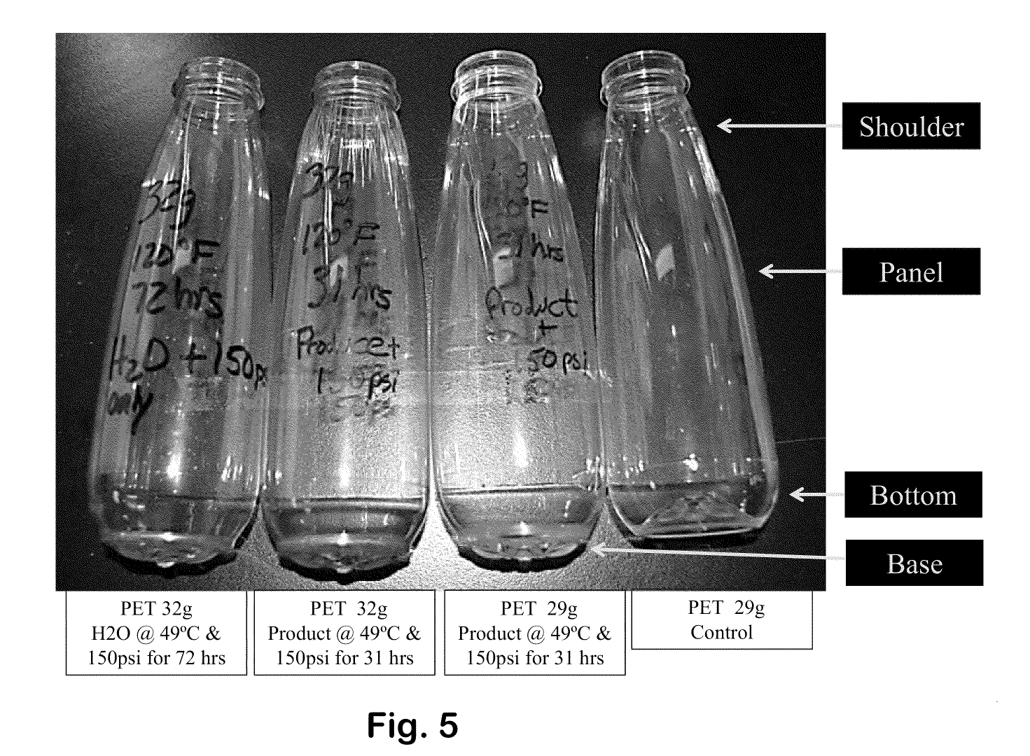

FIG. 5 shows four PET bottles, one being a control, two having contained therein a fragrance-containing product, and one having contained therein tap water, each under certain time, pressure and temperature conditions as noted, to show in the non-control bottles the stress cracks which resulted.

### DETAILED DESCRIPTION OF THE INVENTION

The invention involves a pressurized storage and dispensing system including (1) a plastic aerosol container for storing and dispensing a composition and (2) a composition which includes at least (a) at least one pressurizing component (preferably a propellant such as a compressed gas propellant); (b) at least one active ingredient (preferably at least one fragrance component and, optionally, at least one additional active ingredient); (c) at least one high molecular weight nonionic surfactant; and (d) at least substantially ion-free/salt-free water (preferably ion-free/salt-free water); wherein the composition has a pH of about 4 to about 8.5. Due to storage of the composition in a plastic container, the water component is to be deionized water or other purified water substantially ion-free/salt-free or ion-free/salt-free. Preferably, the composition is also non-flammable. The term “non-flammable” refers to the composition containing less than a total of 1% flammable ingredients and has a flash point above 60° C. (140° F.), or as having been classified as “non-flammable” per CPSC regulations or the Globally Harmonized System of Classification and Labeling of Chemicals (GHS) classification as described above, so as to not require a GHS pictogram, “warning” as a signal word, or the hazard statement of “pressurized container: may burst if heated”, which is the equivalent to a non-flammable aerosol rating. Preferably, the composition also has a volatile organic compound (VOC) content of 0 to about 4% based on the total composition being 100 wt. %; and is under an initial pressure in the pressurized container of about 80 to about 150 psig at 50° C. (122° F.). The water is present in an amount to be the major component of the composition. The plastic container and composition are compatible to provide a stable container and composition combination, i.e., stable in that crazing or the formation of stress cracks in the plastic container is avoided and discoloration of the composition is avoided. The combined container and composition of the invention is suitable for use as a clear container for holding and dispensing a preferably transparent composition. “Clear” is defined as being able to visibly discern media through 1-1.5 cm of solution.

Conventional plastic containers as commercially used for carbonated soda products or non-pressurized products have been unsuitable for storing and dispensing compositions under high pressure as used in aerosol products due to the instability of the plastic containers. This instability under high pressure using conventional plastic container structures has been determined in our work to be based on incompatibility of the composition held under high pressure in the container with the polymer resin of the plastic container. The instability of the container is manifested in crazes or stress cracks formed in portions of the container after filling of the container with a highly pressurized product, e.g., a composition including compressed gas. Crazing is the formation of plastic deformation regions normal to the local tensile strain. The localized plastic deformation of crazing results in formation of fibrils which grow normal to the local tensile strain. Crazing occurs typically in glassy or amorphous type polymers. In glassy thermoplastic polymers, crazes appear to the naked eye as whitened areas due to refraction of light and are visually indistinguishable from cracks. These fibrils can support a portion of the load through the strain hardening. Crazing is generally a precursor to cracking when the strain hardened fibrils fail by breaking under added stress loading. Depending on the nature of induced stress, craze growth typically will result in crack formation and propagation to relieve stress which in turn results in brittle failure of thermoplastic polymers. FIGS. 1, 2 and 3 show by way of example different sizes of cracks. FIG. 1 shows a concentration of micro-cracks in a PET plastic. FIG. 2 shows deeper cracks and FIG. 3 shows yet deeper cracks which extend through the thickness of the PET plastic. The cracks shown in FIG. 3 are sufficiently large for a stored composition to wick there-through.

It has been determined that incompatibility between fragrance-containing pressurized compositions and a plastic container is shown in the creation of crazes and stress cracks. Further, it has been determined that ionic compounds (e.g., salt(s) or ions, such as sodium (Na^{t}) itself or as an element of a compound), present in the composition stored in the plastic body of a container are deposited in these crazes or cracks and influence the growth of crazes or the continued growth of the crazes into cracks and the continued growth of cracks, and failure of the container due to growth in the cracks. FIGS. 4(a)-(d) show a 32 gram PET bottle which had been exposed to a fragrance-containing product including tap water for 31 hours at 49° C. (120° F.) and a pressure of 150 psi. FIG. 4(a) shows stress cracks in the neck area of the PET bottle. FIG. 4(b) shows a magnification of a top portion of the stress cracks in the bottle shown in FIG. 4(a). At this time, the cracks did not extend through the thickness of the wall of the bottle. FIG. 4(c) is a further magnification of a tip of a crack shown in FIG. 4(b). FIG. 4(d) shows a detail of the tip shown in FIG. 4(c). As seen in FIG. 4(d), the crack contains salt deposits, i.e., sodium chloride, which had been present in the tap water making up part of the product contained in the bottle. The sodium will contribute to the crack continuing to grow until the bottle wall fails. FIG. 5 shows four PET bottles. One PET bottle is a control. Of the other bottles, one held tap water for 72 hours at 49° C. (120° F.) under pressure of 150 psi. The other two bottles each held a fragrance-containing product including tap water for 31 hours at 49° C. (120° F.) under a pressure of 150 psi. Stress cracking can be seen in the bottles containing the tap water and the fragrance-containing product.

The pressurized system of the invention includes an active(s)-containing composition with a pressurizing component and plastic aerosol container which avoids crazing and the formation of stress cracks in the plastic container to provide a stable storage and dispensing container and a stable pressurized composition.

Preferred features of the compositions to be stored and dispensed in a plastic aerosol container include the following:

(1) water which is deionized or otherwise treated so that ions and salts are not present in an amount in the water such that the ions or salts are attracted to the surface of a plastic container and serve to attach to such surface and increase the occurrence and growth of crazes and/or stress cracks in the plastic container, i.e., wherein the water is substantially ion-free/salt-free or is ion-free/salt-free;

(2) a mid-range pH of about 4 to about 8.5, preferably about 5 to about 8, and most preferably about 6.5 to about 7.5, since, as shown with PET and believed applicable to other similar or comparable plastics, the composition contains components which at extreme pHs could cleave other bonds (such as ester linkages in PET of a plastic container), e.g., at low pHs serve to protonate oxygen atoms in carbonyl groups and eliminate alcohol which leads to localized reductions in molecular weight via chain cleavage causing craze formations, which can propagate causing brittle bottle failure at high pressure applications, or at high pHs attack carbonyl atoms with hydroxyl ions also which leads to chain cleavage causing craze and/or crack formations resulting in brittle bottle failure under high pressure applications, whereas with a mid-range pH, protons or hydroxyl ions are not present sufficiently to attack either one;

(3) high molecular weight nonionic surfactants, e.g., branched or linear nonionic surfactants having greater than 8 carbon atoms, preferably from 30 to 220 carbon atoms, since it is considered that surfactants tend to move to surfaces (e.g., container walls) which increases the possibility of chemical interaction with the surface and the possibility of crazing and/or cracking, and we have found low molecular weight surfactants tend to attack the plastic of the container, i.e., it is believed, without being bound by theory, that the smaller the molecule attached to the wall of the container, the higher the probability of it diffusing into the polymer and creating damage that results in the craze and subsequent crack, whereas even when absorbed at the surface, high molecular weight surfactants are less likely to diffuse into the polymer and, therefore, less likely to cause damage;

(4) the composition in the container being at least initially pressurized (i.e., when the container is 100% full of the composition as at the time of completion of manufacture) in a range of about 80 to about 150 psig at 50° C. (122° F.)

(5) non-flammable even though the composition is under pressure, i.e., includes less than a total of 1% flammable ingredients (flash point <100° F. (38° C.)) and has an overall composition flash point above 60° C. (140° F.), more preferably has an overall composition flash point of greater than 95° C. (203° F.) so that water vapor can extinguish any flames around 70° C. (158° F.), or “non-flammable” being understood in relation to the CPSC or GHS as described above;

(6) a volatile organic compound (VOC) content of zero to 4%;

(7) is UV stable, which is particularly important since transparent or clear plastic containers are desirable for use, or alternatively, a UV inhibitor can be included in the plastic of the container, in the composition, on the container label, etc. to protect the composition;

(8) the composition is macroscopically single phase which results in a clear composition at room temperature (21° C./70° F.) (“clear” being as defined above) to provide an aesthetically pleasing product to the consumer, whereas by comparison conventional compressed gas air freshening compositions are generally white or cloudy at room temperature; and

(9) includes at least one fragrance ingredient as an active component and, optionally, one or more additional active ingredients.

Features (5) to (9) above while being preferably present to provide an optimally desirable product, are optional in that they are not required to obtain the desired stable pressurized system including a plastic container and active(s)-containing composition.

The composition can include additional components, such as co-surfactant(s), co-solvent(s), pH adjuster(s), and conventional adjuvants as known by one skilled in the art based on the intended use of the composition. Water as described herein is the primary solvent. VOCs (e.g., alcohols, esters, ethers, lactones, etc.) while functional in the composition, are not desirable for inclusion in the composition as a co-solvent since such raise the VOC content of the composition and based on the amount present may make the composition flammable. The components of the composition can be present in amounts suitable for their intended use based on the selected active(s) in the pressurized composition.

When a co-solvent is included, preferably the co-solvent is selected and present in an amount to provide a low or zero VOC content, i.e., a VOC content of less than about 4%. Most preferably, the VOC content is zero, but a VOC-containing co-solvent can be present in a low amount.

As to air or fabric treatment compositions, generally preferred compositions have a formulation within the general formulation as set forth in Table 1 below. The compositions are present as an apparent single phase composition.

TABLE 1

| Ingredients | Wt. % Range |
|---|---|
| Purified Water as described herein | about 80 to about 99 |
| Nonionic Surfactant(s) | about 0.1 to about 2 |
| Active(s) (Fragrance + optional) | about 0.1 to about 2.5 |
| Compressed Gas Propellant(s) | about 0.25 to about 2 |
| pH Adjuster(s) | optionally present in |
| | an amount sufficient |
| | to achieve a pH of |
| | about 4 to about 8.5 |
| Non-Water Solvent(s) | 0 to about 10 |
| Preservative(s) | 0 to about 1 |

Weight percent (wt. %) of the total composition in Table 1 and as used in the description and claims is based on 100 wt. %. The ingredient wt. % given is based on the wt. % of the whole ingredient and not simply on the active(s) of the ingredient.

The water component is at least substantially ion-free/salt-free, is a solvent carrier, and is preferably deionized water. “Substantially ion-free/salt-free” is understood to mean the water is substantially ion-free/salt-free or ion-free/salt-free. Due to storage of the composition in a plastic container, the water is to be deionized or other purified water substantially ion-free/salt-free or ion-free/salt-free. Reverse osmosis water, distilled water, tap water, and/or the like are suitable for use if purified to remove ion(s) and/or salt(s) which may be present therein to at least the extent described. Generally, the water component is present in the composition in an amount greater than about 80 wt. % but less than 100 wt. %. The preferred amount of the water component present is as set forth in Table 1 above. More preferably, the water component is present in an amount of about 90 to about 99 wt. %, and most preferably in an amount of about 92 to about 97.5 wt. %.

Nonionic surfactants suitable for inclusion in the composition are high molecular weight nonionic surfactants which can be branched or linear, saturated or unsaturated, and have greater than 8 carbon atoms, preferably from 30 to 220 carbon atoms, more preferably from 38 to 200 carbon atoms, and most preferably from 64 to 174 carbon atoms. Additionally, the high molecular weight nonionic surfactants preferably have an HLB in a range of about 7 to about 18, more preferably an HLB of about 10 to about 16. For example, preferred high molecular weight nonionic surfactants are branched or linear long chain alkyl-containing nonionic surfactants, saturated or unsaturated, wherein the alkyl group includes from about 8 to about 24 carbon atoms. Further preferred high molecular weight nonionic surfactants are alkyl ethoxylated nonionic surfactants wherein the alkyl group contains from about 16 to about 22 carbon atoms, and from about 7 to 14 moles of ethylene oxide are present. The nonionic surfactants serve to solubilize the fragrance component and/or any other active component(s) present. The solubilization of the fragrance component, and/or other active(s) present, is preferred to be to a degree that a homogeneous solution is provided by the surfactant. Increasing the solubilization of the fragrance component, and/or other active(s) present, serves to decrease the chances of crazing and/or stress cracking.

Suitable nonionic surfactants useful in the pressurized composition include, but are not limited to, polyalkoxylated hydrogenated castor oil, preferably polyethoxylated hydrogenated castor oil such as TAGAT CH60 (60 moles of ethylene oxide (EO)), TAGAT CH40 (40 moles of EO); hydrogenated and ethoxylated castor oil blends, e.g. EUMULGIN HPS (40 moles of EO); secondary alcohol ethoxylates, e.g., certain TERGITOL brand surfactants such as TERGITOL 15-S-7; ethoxylated linear alcohols, e.g., BRIJ 97 (oleth ethoxylate with 10 moles of EO, wherein the C18 alcohol segment includes a double bond), CHEMONIC OE10 (an ethoxylated oleyl alcohol with 10 moles of EO) LUTENSOL brand such as LUTENSOL A08 (8 moles of EO); polyethoxylate sorbitan monooleate (e.g., TWEEN 80); polyoxyethylene sorbitan monolaurate; alkyl polyglycosides; polyethyleneoxide/polypropyleneoxide; alkyl phenol ethoxylated carboxylated alcohols; and mixtures thereof.

The at least one nonionic surfactant is present in an amount range preferably as set forth in Table 1, or in a range of about 0.25 to about 2.0 wt. %, and more preferably in a range of about 0.5 to about 1.0 wt. %, and most preferably about 0.5 to about 0.8 wt. %. The amount of surfactant will vary based on the amount of active and/or fragrance included. The preferred ranges set forth above are based on the active or fragrance being present in an amount of 0.5 wt. %. As the amount of active and/or fragrance is changed, the amount of surfactant will also correspondingly change.

Cationic surfactants can be included in the composition, but are not preferred for inclusion in the composition on the basis of not providing as desirable of results. However, if included such must be in an amount so as to not promote occurrence or growth of crazes or stress cracks. At least one cationic surfactant is present in an amount range of preferably 0 to about 1 wt. %, and more preferably present in an amount of 0 to about 0.5 wt. %. Suitable cationic surfactants for inclusion in the compressed gas composition include, but are not limited to, the following: quaternary ammonium salts, polyoxyethylene alkyl, and mixtures thereof.

Anionic and zwitterionic or amphoteric surfactants are preferably excluded from use in the composition of the invention.

The composition can have a surfactant(s) level which is higher than those generally associated with conventional fragrance-containing compositions. The surfactant to active ingredient(s) preferably is present in a ratio (based on wt. % with the total composition being based on 100 wt. %) of about 1:1, but may be present in a ratio up to or at least about 2.75:1.

Fragrance(s) suitable for inclusion as an active in the aerosol composition can be a natural or synthetic fragrance, based on a single fragrance component or a blend of fragrance components, as well as including solvents, stabilizers, etc., for the fragrance. Fragrances are commercially available from various fragrance manufacturers, such as Firmenich, Givaudan, International Flavors & Fragrances, Inc., Symrise, Takasago, and the like. Other actives useful are as known to one skilled in the art based on the intended use of a composition containing the selected active(s).

The fragrance(s) is(are) present in an amount range generally as set forth above in Table 1 and is(are) preferably present in a range of about 0.1 to about 2.0 wt.%, and more preferably present in a range of from about 0.3 to about 1.0 wt. %.

The pressurized system includes a pressurizing component which preferably is a propellant such as a compressed gas. However, the pressurizing component can also be a non-compressed pressurizing component, such as a bladder pack, piston, or the like. The fluid used with such non-compressed pressurizing components can be either a gas or a liquid to provide the desired pressurizing effect. In addition to possible failure of the plastic container, the use of certain conventional propellants, such as hydrocarbons (e.g., butane, propane and the like) are undesirable as making the composition flammable. Providing a pressurized system including an active(s)-containing composition which preferably is non-flammable (as described above) is an advantage present as a characteristic or feature of the system. The pressurized system, preferably therefore, may consist of any suitable conventionally known compressed gas propellant, including, but not limited to, nitrogen, an inert gas, air, nitrous oxide, carbon dioxide, argon, neon, xenon, or mixtures thereof. Preferred compressed gas propellants are non-oxygen-containing gases since this will further ensure the non-flammable nature of the composition.

The compressed gas propellant is present in an amount generally as set forth above in Table 1, i.e., is preferably present in a range of about 0.25 to about 2 wt. %, and more preferably in a range of about 0.5 to about 1.0 wt. %. The compressed gas propellant is pressurized in a range of from about 80 to about 150 psig at 50° C. (122° F.), as an initial or starting pressure for a 100% full container at the time of completion of manufacture. As the composition is expelled from the container in use, the pressure will drop. When less than 10% volume of the composition remains in the container, the pressure will be in the range of about 10 to about 70 psig at 50° C. (122° F.)

In view of the provision and use of a plastic container for storing and dispensing the active(s)-containing composition, corrosion inhibitor(s) are not necessary for inclusion in the compressed gas composition. In fact, since many corrosion inhibitors are present in salt form, such as potassium dihydrogen phosphate, potassium hydrogen phosphate, diammonium phosphate, potassium phosphate (monobasic or dibasic), sodium phosphate (monobasic or dibasic); nitrites, such as sodium nitrite, potassium nitrite, and ammonium nitrite; silicates, such as sodium meta-silicate; borates; and quaternary ammonium compounds; or ionic forms, such as the nonionic aminomethyl propanol and/or amines, such are not suitable for inclusion in the composition since such salts or ionic compounds as noted would promote occurrence or growth of crazes and/or stress cracks in the plastic container. Conventionally, corrosion inhibitors, such as volatile amines, protect the valve or the headspace.

Non-water solvents or co-solvents suitable for use include glycols, glycol ethers, ketones, esters, lactones, ethers, and carbonates. Alcohols, such as short chain alcohols (e.g., ethanol) can be useful to increase solvation of the active ingredient(s); and to keep components of the composition from depositing on a surface, such as a wall of the plastic container. However, highly volatile organic solvents (acetone, ethanol, dimethyl ester, etc.) are generally not preferred since the inclusion of such in the composition will increase the VOC content and the flammability of the composition, though flammability is not chemistry dependent, but rather volatility dependent and some of these materials, such as acetone and methyl acetate are VOC-exempt. However, if used in an amount of less than 1 wt. % and the composition retains a flash point above 60° C. (140° F.) and has an acceptable VOC content given the composition's intended use, a highly volatile organic solvent can be present. Examples of non-water solvents suitable for use include, but are not limited to, alkylene glycols, such as propylene glycol and triethylene glycol.

The non-water solvent(s) is(are) present in an amount as set forth above in Table 1, i.e., preferably is present in a range of 0 to about 10 wt. %, and more preferably in a range of about 0.1 to about 6.0 wt. %. Most preferably, the non-water solvent is present in an amount of less than or equal to 0.1 wt. % so that the formulation has a low or no flammability.

A pH adjuster is optional. However, if a pH adjuster is included, suitable compounds for inclusion in the pressurized composition as a pH adjuster or controller must be nonionic in nature. pH adjusters, such as caustic soda, lactic acid, citric acid, etc. are not desirable for inclusion. Since the pH is not critical for corrosivity reasons, pH adjusters are not desirable for inclusion in the composition.

The pH adjuster when present is used in an amount sufficient to obtain a pH in a range of about 4 to about 8.5, more preferably to obtain a pH in a range of about 5 to about 8, and most preferably to obtain a pH in a range of about 6.5 to about 7.5.

Preservative(s) suitable for inclusion in the pressurized composition include, but are not limited to isothiazolinones, such as 2-methyl-4-isothiazolin-3-one, which is sold under the trade name NEOLONE M-10; or any suitable preservative know in the art that is preferably nonionic.

The preservative(s) is(are) present in an amount as generally set forth above in Table 1, preferably in an amount in the range of about 0.01 to about 1.0 wt. %, more preferably in a range of about 0.01 to about 0.5 wt. %, and most preferably in a range of about 0.05 to about 0.2 wt. %. Ultimately, the range depends on the preservative choice, pH, and other factors.

Dispensing containers suitable for use can be essentially any type of container having a body for storing safely a composition under pressure to be dispensed and a spray head or spray nozzle for receiving the composition from the body and dispensing the received composition through a spray orifice in the spray head or nozzle to disperse the composition as a plurality of aerosol particles. Preferred dispensing containers in view of the invention are conventional non-piston aerosol containers including a plastic body for containing the composition to be dispensed. The plastic body must be capable of withstanding operating pressure of the composition, preferably the resin is capable of withholding pressure in a range of about 70 to about 180 psig. The spray head or nozzle can be as conventionally available for providing a spray dispersion of a composition maintained under pressure. Plastic containers may include bodies made of branched or linear PET (polyethylene terephthalate), PEN (polyethylene naphthalate); PEF (polyethylene furanoate), PC (polycarbonate), polyolefins, such as polyethylene and polypropylene; and other polyesters, and blends thereof, as well as generally other plastics and blends thereof known for use. PET resins and blends thereof with intrinsic viscosity (IV) values greater than 0.75 dL/g (deciliters per gram) (ASTM D 4603), more preferably greater than 0.80 dL/g, and most preferably greater than 0.85 dL/g, are more resistant to craze initiation and propagation that leads to crack formation. Additionally, branched PET resins are more resistant to crazing or crack formation than linear PET resins. Further, annealing the plastic body of the container reduces the creep rate, but does not eliminate it. Annealing may take place at or below 65° C. (149° F.) for 2 hours or more depending on the annealing temperature. Annealing below 65° C. may require longer annealing time to reduce creep rate. The dispensing spray head or nozzle for the container can be selected based on the spray rate and particle size desired to be provided based on the invention described herein.

Another way to determine compatibility between the active(s)-containing composition and plastic container is based on Hansen Solubility Parameters (HSP) of the composition in relation to the plastic (polymer resin) of the container.

According to Hansen, any material can be characterized by three parameters: σ_{d}, σₚ, σₕ, wherein σ_{d} characterizes the energy from dispersion forces between molecules, σₚ corresponds to the energy from dipolar intermolecular force between molecules, and σₕ corresponds to the energy from hydrogen bonds between molecules. These three parameters can be conveniently used as coordinates in three-dimensional space.

Any material can then be represented by a point in such space. It is proposed that a distance between two points in this space be used to predict the strength of interactions between the materials represented by these points, with short distances corresponding to strong interactions and long distances corresponding to weak or no interactions.

To calculate the distance (Rₐ) between points in Hansen space, the following formula is used:

(Rₐ)²=4(σ_{d2}−σ_{d1})²+(σₚ₂−σₚ₁)²+(σₕ₂−σₕ₁)²

wherein the indices 1 and 2 refer to the two materials being evaluated. Combining this with a predetermined interaction radius (R_{O}) gives the relative energy difference (RED) of the system : RED=Rₐ/R_{O}. RED is indicative as follows: where RED<1, the molecules are likely to interact (mix or swell or craze); where RED=1, the system will partially interact; and where RED>1, the system will not interact (mix or craze). In the present invention, neither the composition nor its components should dissolve into the polymer of the plastic container. The components of the composition (e.g., water serving as a solvent carrier and optional co-solvent(s)) of the invention should have a RED value of about 1.5 to about 25, more preferably about 3 to about 25, and most preferably about 5 to about 25. For the present invention, the polymer resin of the container and the at least substantially anion-free/salt-free water of the composition preferably have a HSP with a distance value (Rₐ) of about 10 to about 35.

The benefits achieved as described above in providing a compressed gas pressurized active(s)-containing composition stored and dispensed from a plastic container are significant and the spray head or spray nozzle utilized with the container can be selected to provide dispensing at a spray rate and/or particle size a conventionally know. However, if desired to add to the above described benefits, the properties of the active(s) present in the composition can be further enhanced by providing in combination at least

(1) a spray rate for dispensing the composition which is greater than about 1.5 to about 3.0 grams/second (g/s), preferably about 1.6 to about 2.5 g/s and further about 1.7 to about 2.2 g/s, wherein the spray rate is determined by measuring the rate of product expelled by an initial spray of product for a 60 second duration of a 100% full container of product (this spray duration resulting in dispensing of approximately one half of a 120 ml aerosol container); and

(2) particle size of the composition during dispensing is maintained to provide an average particle size of about 60 to about 100 microns, preferably about 60 to about 90 microns, more preferably about 60 to about 80 microns. Particle size was measured using a particle analyzer as manufactured by Malvern Instruments, Inc., Model STP5311, which is described as the Short Bench Spraytec Laboratory system with 950 mm optical bench and 300 mm lens. Samples are spray tested at a central plane, 9 inches from the beam utilizing programmed software of the Malvern particle analyzer to obtain mass median diameter (MMD) for a particle diameter in which 50% of the volume sampled is below the measured particle diameter. The measured results are provided for 10 seconds and 60 seconds.

The pressure within the container is at least initially or starts in a range of from about 80 to about 150 psig at 50° C. (122° F.) (all pressure measurements herein being understood to be at 50° C.)

The spray rate range of (1) above ensures an ideal amount of active(s) is(are) released from the container. The active(s)-containing compressed gas composition is provided in a dispensing container for storing and dispensing the composition as an aerosol spray. The ratio of compressed gas to composition is preferably about 50:50 to about 25:75 by volume. As set forth above, the compressed gas can include compressed air, nitrogen, nitrous oxide, an inert gas, carbon dioxide, argon, neon, xenon, or combinations thereof. The compressed gas preferably is non-oxygen-containing to enhance the non-flammable feature of the composition and, thus, the overall product. The active(s)-containing composition is to be released at a spray rate of greater than about 1.5 g/s to about 3.0 g/s, wherein the spray rate is determined as set forth above in (1), i.e., 60 second expulsion from a 100% full container for a 120 ml container. The average initial particle size of the spray is about 60 to about 100 microns, preferably about 60 to about 90 microns, and more preferably about 60 to about 80 microns. The active fragrance component of the composition can be one or more conventional natural or synthetic perfuming or fragrance compound. Optional additional active ingredients may be included, such as odor elimination compound(s) such as triethylene glycol (TEG), insecticide(s), disinfectant(s), antimicrobial(s), or the like.

Provision of the described particle size in combination with the described spray rate results in enhanced fragrancing and odor elimination, when the active is a fragrance or odor eliminator, upon dispersal of the fragrance and/or odor treating composition. The size of the particles allows for longer residence time in the treated air. The longer residence time in the air improves the fragrance experience of the consumer with respect to maintaining the character of the fragrance over a longer period of time.

The maintaining or control of the pressure within the range described above ensures sufficient breakup of the liquid fragrance or odor treating composition as it is dispensed from a container. Particle size of the aerosol can be affected by the pressure of the aerosol, especially with increased product usage.

While the invention is described primarily with respect to compositions for treating air, e.g., fragrancing and/or odor elimination, the invention is also applicable to fragrancing fabric or other surfaces or eliminating odors on fabric or other surfaces. Compressed gas aerosols are advantageous in treating fabrics since such provide for a good evaporation rate from fabric and avoid resoiling following treatment. The evaporation rate results in a longer life of the product in use.

Examples of compressed gas formulations of the invention for storing and dispensing in a plastic aerosol container are set forth below. Preferred plastic for the containers for storing and dispensing such formulations is PET, PEN, blends of PET and PEN, or copolymers of PET-PEN resins.

FORMULA 1

| INGREDIENTS | WT. % |
|---|---|
| Deionized Water | 97.45 |
| 2-Methyl-4-Isothiazolin-3-One | 0.15 |
| (9.9%) | |
| (e.g., NEOLONE M-10) | |
| Propylene Glycol | 0.47 |
| Secondary Alcohol Ethoxylate (7 EO) | 0.28 |
| (100%) (Nonionic) | |
| (e.g., TERGITOL 15-S-7) | |
| Hydrogenated Castor Oil Ethoxylate | 0.47 |
| (60 EO) | |
| Fragrance (various) | 0.50 |
| Nitrogen Propellant | 0.68 |
| Total | 100 |

FORMULA 2

| INGREDIENTS | WT. % |
|---|---|
| Deionized Water | 97.15 |
| 2-Methyl-4-Isothiazolin-3-One | 0.15 |
| (9.9%) | |
| (e.g., NEOLONE M-10) | |
| Propylene Glycol | 0.47 |
| Secondary Alcohol Ethoxylate (7 EO) | 0.39 |
| (100%) (Nonionic) | |
| (e.g., TERGITOL 15-S-7) | |
| Hydrogenated Castor Oil Ethoxylate | 0.66 |
| (60 EO) | |
| Fragrance (various) | 0.50 |
| Nitrogen Propellant | 0.68 |
| Total | 100 |

FORMULA 3

| INGREDIENTS | WT. % |
|---|---|
| Deionized Water | 95.87 |
| 2-Methyl-4-Isothiazolin-3-One | 0.15 |
| (9.9%) | |
| (e.g., NEOLONE M-10) | |
| Oleth Ethoxylate (10 EO) | 1.13 |
| Nonionic | |
| (e.g., BRIJ 97) | |
| Hydrogenated Castor Oil Ethoxylate | 1.67 |
| (60 EO) | |
| Fragrance (various) | 0.50 |
| Nitrogen Propellant | 0.68 |
| Total | 100 |

FORMULA 4

| INGREDIENTS | WT. % |
|---|---|
| Deionized Water | 96.96 |
| 2-Methyl-4-Isothiazolin-3-One | 0.15 |
| (9.9%) | |
| (e.g., NEOLONE M-10) | |
| Propylene Glycol | 0.66 |
| Ethoxylated Linear Alcohol (7 EO) | 0.39 |
| Hydrogenated Castor Oil Ethoxylate | 0.66 |
| (60 EO) | |
| Fragrance (various) | 0.50 |
| Nitrogen Propellant | 0.68 |
| Total | 100 |

FORMULA 5

| INGREDIENTS | WT. % |
|---|---|
| Deionized Water | 97.15 |
| 2-Methyl-4-Isothiazolin-3-One | 0.15 |
| (9.9%) | |
| (e.g., NEOLONE M-10) | |
| Propylene Glycol | 0.47 |
| Ethoxylated Linear Alcohol (7 EO) | 0.28 |
| Hydrogenated Castor Oil Ethoxylate | 0.77 |
| (60 EO) | |
| Fragrance (various) | 0.50 |
| Nitrogen Propellant | 0.68 |
| Total | 100 |

FORMULA 6

| INGREDIENTS | WT. % |
|---|---|
| Deionized Water | 97.17 |
| 2-Methyl-4-Isothiazolin-3-One | 0.15 |
| (9.9%) | |
| (e.g., NEOLONE M-10) | |
| Propylene Glycol | 0.47 |
| Ethoxylated Linear Alcohol (7 EO) | 0.56 |
| Hydrogenated Castor Oil Ethoxylate | 0.47 |
| (60 EO) | |
| Fragrance (various) | 0.50 |
| Nitrogen Propellant | 0.68 |
| Total | 100 |

FORMULA 7

| INGREDIENTS | WT. % |
|---|---|
| Deionized Water | 97.152 |
| 2-Methyl-4-Isothiazolin-3-One | 0.15 |
| (9.9%) | |
| (e.g., NEOLONE M-10) | |
| Propylene Glycol | 0.47 |
| Ethoxylated Linear Alcohol (7 EO) | 0.39 |
| Hydrogenated Castor Oil Ethoxylate | 0.66 |
| (60 EO) | |
| Fragrance (various) | 0.50 |
| Nitrogen Propellant | 0.68 |
| Total | 100 |

FORMULA 8

| INGREDIENTS | WT. % |
|---|---|
| Deionized Water | 90.87 |
| 2-Methyl-4-Isothiazolin-3-One | 0.15 |
| (9.9%) | |
| (e.g., NEOLONE M-10) | |
| Ethyl Alcohol SDA-40B 200 Proof | 5.00 |
| Oleth Ethoxylate (10 EO) | 1.13 |
| Nonionic | |
| (e.g., BRIJ 97) | |
| Hydrogenated Castor Oil Ethoxylate | 1.67 |
| (60 EO) | |
| Fragrance (various) | 0.50 |
| Nitrogen Propellant | 0.68 |
| Total | 100 |

The exemplary embodiments herein disclosed are not intended to be exhaustive or to unnecessarily limit the scope of the invention. The exemplary embodiments were chosen and described in order to explain the principles of the present invention so that others skilled in the art may practice the invention. As will be apparent to one skilled in the art, various modifications can be made within the scope of the aforesaid description. Such modifications being within the ability of one skilled in the art form a part of the present invention and are embraced by the appended claims.

It is claimed:

1. A pressurized article comprising
(a) a pressurized container for storing and dispensing a composition, wherein said container includes a spray nozzle and a plastic body for containing said composition; and
(b) said composition for storing in and dispensing from said container comprising
(i) at least one pressurizing component;
(ii) at least one active ingredient;
(iii) at least one high molecular weight nonionic surfactant having greater than 8 carbon atoms;
(iv) water as a solvent carrier in an amount to serve as a major component of said composition,
wherein said water is at least substantially ion-free and at least substantially salt-free;
wherein said composition has a pH of about 4 to about 8.5, and is present in said container under an initial pressure of about 80 to about 150 psig at 50° C. (122° F.).

2. The pressurized article of claim 1, wherein said plastic of said body of said container is selected from polyethylene terephthalate (PET), polyethylene naphthalate (PEN), polyethylene furanoate (PEF), polycarbonate (PC), polyolefins, or a blend thereof, and said plastic is structured to withhold pressure of the composition.

3. The pressurized article of claim 1, wherein said at least one high molecular weight nonionic surfactant has from 30-220 carbon atoms.

4. The pressurized article of claim 1, wherein said at least one pressurizing component is a non-flammable propellant, or a non-compressed pressurizing element.

5. The pressurized article of claim 1, wherein said at least one pressurizing component is a compressed gas propellant.

6. The pressurized article of claim 5, wherein said compressed gas propellant is one or more of nitrogen, air, carbon dioxide, nitrous oxide, argon, neon, xenon, an inert gas, and blends thereof.

7. The pressurized article of claim 1, wherein said at least one active ingredient is one or more of a fragrance component, an odor eliminating compound, an insecticide, an antimicrobial, and a disinfectant.

8. The pressurized article of claim 1, wherein said spray nozzle is configured to dispense said composition at an initial spray rate in a range of from greater than about 1.5 grams/second (g/s) to about 3.0 g/s, and has an initial average particle size in a range of up to about 100 microns.

9. The pressurized article of claim 8, wherein said initial spray rate is in a range of from about 1.6 to about 2.5 g/s, and said initial average particle size is in a range of about 60 to about 90 microns.

10. The pressurized article of claim 1, wherein said at least one pressurizing component is present in an amount of about 0.25 to about 2 wt. %; said at least one active ingredient is present in an amount of about 0.1 to about 2.5 wt. %; said at least one high molecular weight nonionic surfactant is present in an amount of about 0.1 to about 2 wt. %; and said water is present in an amount of about 80 to about 99 wt. %; wherein the total composition is based on 100 wt. %.

11. A pressurized article for fragrance dispensing and/or odor treating comprising
(1) a composition comprising
(a) at least one compressed gas propellant;
(b) at least one active ingredient including at least one fragrance component and, optionally, at least one additional active ingredient;
(c) at least one high molecular weight nonionic surfactant having about 30 to about 220 carbon atoms; and
(d) water as a solvent carrier and in an amount to serve as a major component of said composition wherein said water is at least substantially ion-free and at least substantially salt-free;
wherein said composition has a pH of about 4 to about 8.5, and has a VOC content of zero to 4%;
(2) a pressurized dispensing container adapted to contain and dispense said composition wherein said composition has
(i) an initial pressure in said container of about 80 to about 150 psig at a temperature of 50° C. (122° F.); and
(ii) said aerosol dispensing container includes a body for holding said composition and said body is made of plastic.

12. The article of claim 11, wherein said plastic is PET, PEN, PEF, PC, polyolefins, or a blend thereof, and said plastic is structured to withhold pressure of the composition.

13. The article of claim 11, wherein said at least one compressed gas propellant is one or more of air, nitrogen, carbon dioxide, nitrous oxide, argon, neon, xenon, an inert gas, and blends thereof, and said plastic is structure to withhold pressure of the composition.

14. The article of claim 11, wherein said at least one active ingredient is one or more of an odor eliminating compound, an insecticide, an antimicrobial, and a disinfectant.

15. The article of claim 11, further comprising a spray nozzle which dispenses said composition at an initial spray rate of greater than about 1.5 g/s to about 3.0 g/s and in an initial average particle size of up to about 100 microns.

16. The article of claim 15, wherein said initial spray rate is from about 1.6 g/s to about 2.5 g/s.

17. The article of claim 15, wherein said initial average particle size is from about 60 to less than 90 microns.

18. The article of claim 11, wherein said at least one compressed gas propellant is present in an amount of about 0.25 to about 2 wt. %; said at least one active ingredient is present in an amount of about 0.1 to about 2.5 wt. %; said at least one high molecular weight nonionic surfactant is present in an amount of about 0.1 to about 2 wt. %; and said water is present in an amount of about 80 to about 99 wt. %; wherein the total composition is based on 100 wt. %.

19. A pressurized plastic article comprising
(a) a pressurized container for storing and dispensing a composition, wherein said container includes a spray nozzle and a body composed of polymer resin for containing said composition, said polymer resin being structured to withhold working pressure of the composition; and
(b) said composition for storing in and dispensing from said container comprising
(i) at least one compressed gas propellant;
(ii) at least one active ingredient;
(iii) at least one high molecular weight nonionic surfactant having greater than 8 carbon atoms;
(iv) water as a solvent carrier in an amount to serve as a major component of said composition, wherein said water is at least substantially ion-free and at least substantially salt-free;
wherein said composition has a pH of about 4 to about 8.5, is present in said container under an initial pressure of about 80 to about 150 psig at 50° C. (122° F.), and wherein said polymer resin and said water has a Hansen Solubility Parameters with a distance value (Rₐ) of about 10 to about 35.

20. The pressurized plastic article of claim 19, wherein said at least one high molecular weight nonionic surfactant has from 30-220 carbon atoms.

21. The pressurized plastic article of claim 19, wherein said at least one compressed gas propellant is one or more of air, nitrogen, carbon dioxide, nitrous oxide, argon, neon, xenon, an inert gas and blends thereof.

22. The pressurized plastic article of claim 19, wherein said at least one active ingredient is one or more of a fragrance component, an odor eliminating compound, an insecticide, an antimicrobial, and a disinfectant.

23. The pressurized plastic article of claim 19, wherein said composition is dispensed from said plastic body by said spray nozzle at an initial spray rate upon dispensing of greater than about 1.5 to about 3.0 grams/second (g/s), and with an initial average particle size in a range of up to about 100 microns.

24. The pressurized plastic article of claim 23, wherein the initial spray rate is about 1.6 g/s to about 2.5 g/s and wherein the initial average particle size is about 60 to about 90 microns.

25. The pressurized plastic article of claim 19, wherein the plastic of the container is selected from PET, PEN, PEF, PC, polyolefins, and blends thereof.

26. A method of dispensing a fragranced composition comprising dispensing said fragranced composition from a pressurized dispensing container including a spray nozzle and a body made of plastic; wherein said composition is dispensed under an initial pressure in said container of about 80 to about 150 psig at 50° C. (122° F.); wherein said composition comprises (a) at least one compressed gas propellant, (b) at least one active ingredient, (c) at least one high molecular weight nonionic surfactant having from about 30 to about 220 carbon atoms; and (d) water as a solvent carrier and in an amount to serve as a major component of said composition, wherein said water is at least substantially ion-free and at least substantially salt-free; and wherein said composition has a pH of about 4 to about 8.5.

27. The method of claim 26, wherein said plastic is PET, PEN, PEF, PC, polyolefins, or a blend thereof, and said plastic is structured to withhold working pressure of the composition.

28. The method of claim 26, wherein said at least one active ingredient is one or more of a fragrance component, an odor reducing compound, an insecticide, an antimicrobial, and a disinfectant.

29. The method of claim 26, wherein said composition is dispensed from said container through said spray nozzle at an initial spray rate upon dispensing of greater than about 1.5 grams/second (g/s) to about 3.0 g/s, and with an initial average particle size in a range of up to about 100 microns.

30. The method of claim 26, wherein said at least one compressed gas propellant is present in an amount of about 0.25 to about 2 wt. %; said at least one active ingredient is present in an amount of about 0.1 to about 2.5 wt. %;
said at least one high molecular weight nonionic surfactant is present in an amount of about 0.1 to about 2 wt. %; and
said water is present in an amount of about 80 to about 99 wt. %; wherein the total composition is based on 100 wt. %.
